(19) United States
(12) Patent Application Publication
(10) Pub. No.: US 2023/0248461 A1
(43) Pub. Date: Aug. 10, 2023

(54) TISSUE EXPANSION DEVICE WITH FLUID COLLECTION

(71) Applicant: Allergan Inc., Irvine, CA (US)

(72) Inventor: David J. Schuessler, Santa Ana, CA (US)

(21) Appl. No.: 18/015,136

(22) PCT Filed: Jul. 9, 2021
(86) PCT No.: PCT/US2021/041187

§ 371 Date: Jan. 9, 2023

Related U.S. Application Data
(60) Provisional application No. 63/050,173, filed on Jul. 10, 2020.

(51) Int. Cl. A61B 90/00; A61M 27/00

(52) U.S. Cl. CPC A61B 90/02; A61B 90/08; A61M 27/00; A61B 2090/0815; A61M 2205/04; A61M 2210/1007

(57) ABSTRACT

A tissue expander can be configured to collect extracellular fluid from within a patient’s body cavity. The tissue expander can be implanted into a subdermal cavity during a procedure, such as a breast reconstruction procedure, and facilitate the expansion of the subdermal cavity in preparation for placement of a final implant.

### CROSS-REFERENCE TO RELATED APPLICATIONS

The present application claims the benefit of U.S. Provisional Application No. 63/050,173, filed Jul. 10, 2020, the entirety of which is incorporated herein by reference.

### BACKGROUND

### Field

The present disclosure relates to prosthetic implants and, more particularly, to breast implants and tissue expanders that incorporate a fluid collection feature or component.

### Description of the Related Art

Implantable prostheses are commonly used to replace or augment body tissue. These implants serve to support surrounding tissue and to maintain the appearance of the body. The restoration of the normal appearance of the body has an extremely beneficial psychological effect on post-operative patients, alleviating much of the shock and depression that often follows extensive surgical procedures. In the case of breast cancer, a mastectomy is sometimes necessary to remove some or all of the mammary gland and surrounding tissue, which creates a void. This void may be filled with a fluid-filled implantable prosthesis.

Prior to implantation of a long-term prosthesis, it is common practice to utilize a more temporary implant, for example, what is known as a “tissue expander” in order to gradually create the space necessary for the long-term prosthesis. Tissue expander devices can be used to expand the skin and overlying tissues of a skin defect or where additional skin and tissue are needed for repair or reconstruction. For example, in some situations, such as a mastectomy, the chest tissues may be flat and tight, and an expander can serve to prepare the body for receiving a long-term prosthesis. Tissue expanders can also be used in other places in the body to expand healthy tissue to replace a nearby defect such as a burn or scar. Typically, a tissue expander comprises an inflatable body, having an inflation valve connected thereto. The valve may be formed into the inflatable body itself or may be remotely located and connected to the inflatable body by means of an elongated conduit.

The inflatable body of the tissue expander is placed subcutaneously in the patient, at the location of where tissue is to be expanded. The inflation valve or injection port, whether on the implant or remote thereto, is also subcutaneously positioned or implanted, and is configured to allow gradual introduction of fluid, typically saline, into the inflation body, by injection with a syringe. After gradual inflation at pre-determined intervals, the skin and subcutaneous tissues overlying the expander are caused to expand in response to the pressure exerted upon such tissues by the inflatable body as solution is gradually introduced therein.

One of the side effects of almost any invasive surgery is the damage caused to the dissected tissue and the resultant extracellular fluid egress into the surrounding area over the subsequent days. In case of breast reconstruction, for example, temporary drains may be used to remove the excess fluid. The surgery may create a significant effusion of fluid from the surrounding tissues that may not be resorbed by the lymphatic system. If the fluid is not removed during the post-operative period, it can create swelling, produce a seroma, and/or be a source of pain and infection.

### SUMMARY

In accordance with some of the embodiments disclosed herein is the realization that there is a need to provide an effective drain to collect excess fluid during a reconstruction procedure. For example, in some embodiments, a drainage device can be provided near the periphery of the tissue expander, e.g., in an inferior position near the inframammary fold in case of a breast reconstruction surgery, where excess fluid can collect. The fluid collected by the drain may be removed by suctioning it out from an end that is outside the body, e.g., through a puncture site in the lateral side of the chest. Alternately, the end of the drain may be provided with a self-sealing injection port that could remain implanted in the lateral chest area. The accumulated fluid could then be periodically removed by manual aspiration with a syringe or other means.

After implantation, a drainage device may be left in the tissue expansion site, for example, for one or two weeks until the healing process reduces fluid creation to a rate that the lymphatic system can maintain. The drain can then be removed by pulling the external end from the original puncture would, leaving the tissue expander in place. Alternately, the drain may be cut at or near the puncture site, and the wound closed, leaving the remainder of the drain in place until the tissue expander is removed.

In some embodiments, the present disclosure provides a tissue expander (also referred to herein as a drainage device) that can address various disadvantages from prior drainage devices and provide numerous advantages and benefits to patients and clinicians. For example, prior drainage devices can become clogged, rendering them ineffective. Additionally, prior drainage devices and any attached reservoirs are inconvenient and uncomfortable for the patient and can limit the patient’s activities. Further, prior drainage devices themselves can act as potential source of infection by providing a conduit for bacteria from outside the body to the surgical wound and device site which can lead to infection and biofilm formation.

In accordance with some embodiments disclosed herein is the realization that effective treatment to minimize or address seroma formation may be most effective if a fluid is collected or absorbed directly by or into the tissue expander itself after implantation into the patient. The fluid absorption mechanism may absorb and retain extracellular fluid adjacent to the tissue expander upon implantation of the tissue expander until the tissue expander is removed from the body.

In some embodiments, the tissue expander can comprise an expandable shell having an anterior portion, a posterior portion and a perimeter. The tissue expander may include a fluid absorption or collection component.

The fluid collecting component can be coupled, attached, integral with (such as a single, continuous form, material, or part), or connected to any portion or structure of the tissue expander.

For example, the tissue expander may include a fluid collection component, which can be coupled to the anterior portion, the posterior portion, and/or the perimeter thereof. At least a portion of the posterior portion may comprise a laminate including the fluid collection component and a tear-resistant material.

Optionally, the laminate may form a ring round the posterior portion of the tissue expander. The ring may be configured to wick away the extracellular fluid from the tissue adjacent the tissue expander.

In some embodiments, the fluid collection component may include a fibrous mesh. In some embodiments, the fluid collection component may include an open cell matrix foam.

In some embodiments, the fluid collection component may include an expandable elastomeric matrix and granules of a solute embedded within the matrix. In some embodiments, the expandable elastomeric matrix may include a foamed elastomer. In some embodiments, the expandable elastomeric matrix may include silicone. The solute may include sodium chloride in some embodiments. In some embodiments, the expandable elastomeric matrix defines boundaries of a plurality of chambers within the matrix. In some embodiments, the plurality of chambers is fluidly interconnected as open cells of the matrix.

In some embodiments, the tissue expander may further include features to adapt to an acellular dermal matrix (ADM).

In accordance with some embodiments, a method of reducing an amount of extracellular fluid adjacent a tissue expander upon implantation of the tissue expander may include positioning the tissue expander having a posterior portion within a subdermal cavity such that the posterior portion contacts an inferior portion of the subdermal cavity and the extracellular fluid from the inferior portion of the subdermal cavity is absorbed into a fluid collection component of the posterior portion.

In accordance with some embodiments, a method of expanding a soft tissue in a body of a living subject may include forming a subdermal cavity at a site where the soft tissue is to be expanded. A tissue expander can be implanted in the subdermal cavity. The tissue expander includes an expandable shell having an anterior portion, a posterior portion and a perimeter. The posterior portion includes a fluid collection component configured to absorb extracellular fluid adj acent the tissue expander upon implantation.

In some embodiments, implanting the tissue expander may include positioning the tissue expander within the subdermal cavity such that the posterior portion contacts an inferior portion of the subdermal cavity and extracellular fluid from the inferior portion of the subdermal cavity is absorbed into the fluid collection component. A portion of the perimeter may be sutured to the inferior portion of the subdermal cavity in some embodiments. Upon implantation of the tissue expander, the expandable shell may be expanded to expand the soft tissue. In some embodiments, the expandable shell may be expanded by filling the expandable shell with a fluid.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1 is a front plan view of a tissue expander having a fluid collection component, according to some embodiments of the present disclosure.

FIG. 2 is a side view of the tissue expander of FIG. 1.

FIG. 3 is a cross-section view of a fluid collection component for a tissue expander, according to some embodiments of the present disclosure.

FIG. 4 is a cross-sectional view through a fluid collection component, according to some embodiments of the present disclosure.

FIG. 5 is a cross-sectional view through a fluid collection component, according to some embodiments of the present disclosure.

FIG. 6 is a front plan view of a suture rim for a soft tissue expander, according to some embodiments of the present disclosure.

FIG. 7 is a front plan view of another embodiment of a tissue expander having a fluid collection component in the form of a plurality of suture tabs, according to some embodiments of the present disclosure.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

As described above, although tissue expanders can be used in breast reconstruction procedures, certain risks arise may their use. In use, a properly placed drainage device can enable a clinician to facilitate removal of extracellular fluid accumulating in the subdermal cavity where a tissue expander is implanted. Further, the present Applicant has developed unique and innovative tissue expanders that can include a drainage or collection tube where the extracellular fluid can be collected and an access port for removing the collected extracellular fluid. For example, the Applicant has developed devices and systems such as those disclosed in U.S. Provisional Pat. App. No. 63/015,403, the entirety of which is incorporated herein by reference.

The present disclosure provides various embodiments of a tissue expander having a fluid collection component to facilitate removal and isolation of extracellular fluid from the tissues adjacent to the tissue expander. The fluid collection component can help reduce fluid accumulation and mitigate against seroma formation, as well as reduce or eliminate the need for drains or manual post-operative fluid management.

In accordance with some embodiments, a tissue expander can be configured to include an expandable shell and a fluid collection component coupled to or connected with the expandable shell. The fluid collection component can be configured to absorb extracellular fluid adjacent the tissue expander upon implantation.

Further, in accordance with some embodiments, the present disclosure relates to a tissue expander that is configured to absorb and/or collect extracellular fluid within a body cavity. The tissue expander can be configured to carry the absorbed or collected extracellular fluid. Optionally, the tissue expander can bodily incorporate the extracellular fluid to enable removal of both the tissue expander and the extracellular fluid at a desired time.

Rather than requiring an additional operation to withdraw fluid or to insert a drain subcutaneously after a seroma has formed, some embodiments of the tissue expanders disclosed herein can begin working to remove fluid from the body cavity immediately upon implantation by absorbing or collecting such fluid from the body cavity for improving patient comfort and recovery.

The fluid can be isolated or captured by and/or within one or more components of the tissue expander, obviating the need for a drainage structure which may increase the risk of infections. Moreover, the fluid can be stored or carried by such component(s) of the tissue expander and removed at a later time, such as when the tissue expander is removed from the body.

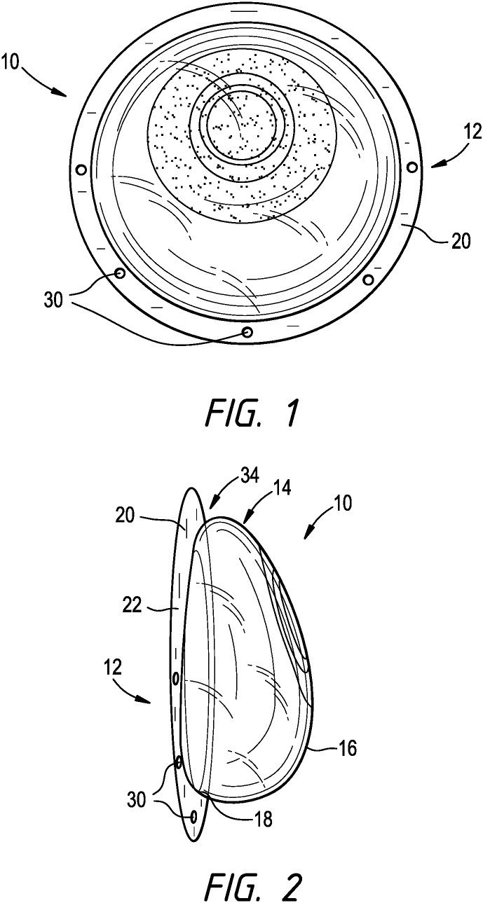

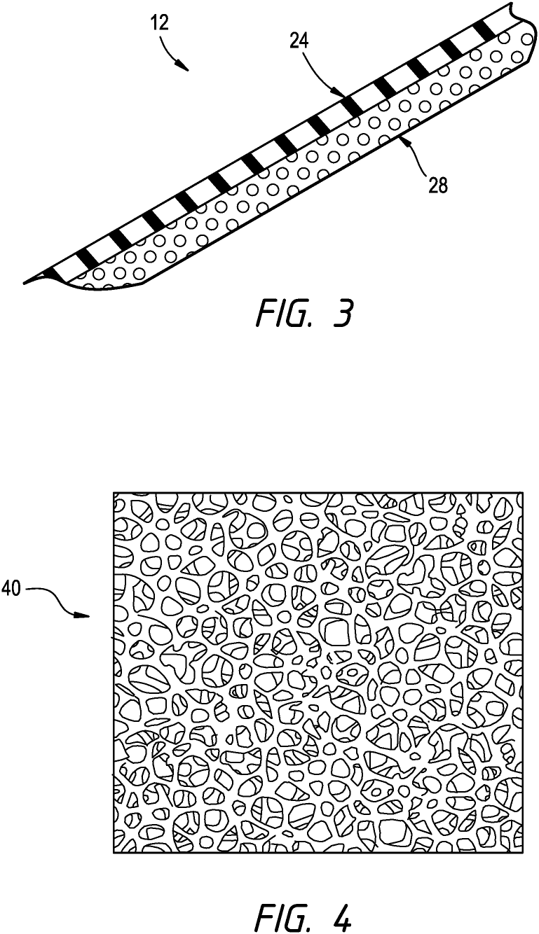

For example, referring to FIGS. 1 and 2, some embodiments disclosed herein relate to an improvement to a tissue expander 10 that comprises an expandable shell 14 having an anterior portion 16 and a posterior portion 18.

The tissue expander can further include a base 12 coupled to the posterior portion 18 of the expandable shell 14. In some embodiments, the base 12 can comprise a suture strip or rim 20 that is coupled to a posterior portion 18 of the expandable shell 14.

In some embodiments, the rim 20 of the base 12 can comprise one or more apertures 30 which can be designed to accommodate or support a drainage structure such as, for example, drain tube (not shown). When implanted, the posterior portion 18 can be positioned such that the base 12 is in contact with an inferior portion of the subdermal cavity, e.g., near the inframammary fold in case of a breast reconstruction, where excess fluid can collect.

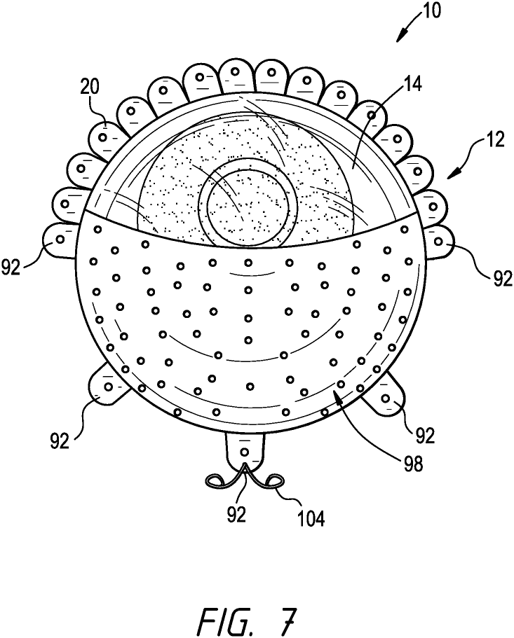

As shown in FIGS. 1 and 2, the base 12 can comprise a single circumferential flange that extends partially or fully around a perimeter of the tissue expander 10. In some embodiments, the base 12 can define a larger diametric or top-view dimension, as seen in the top view of FIG. 1, than a diametric or top-view dimension of the expandable shell 14 of the tissue expander 10. The base 12 can also comprise multiple tabs, as illustrated in the embodiment shown in FIG. 7. Advantageously, these tabs can each be independently moved to extended or stowed positions in order to facilitate fluid absorption and locations adjacent to or spaced apart from the tissue expander 10.

Referring now to FIG. 3, in some embodiments, one or more portions of the base 12 may comprise a laminate structure. In some embodiments, the laminate structure of the base 12 can comprise a tear-resistant material component 24. Further, in some embodiments, the base 12 can also comprise a fluid collection component 28. The laminate structure for the base 12 can comprise one or more layers of the tear-resistant material component 24 and one or more layers of the fluid collection component 28.

The embodiment illustrated in FIG. 3 shows a laminate structure for the base 12 that comprises a tear-resistant material component 24 and a fluid collection component 28. Thus, in some embodiments, the base can comprise a “sandwich” of two layers of a tear-resistant material component 24 and a fluid collection component 28 interposed therebetween.

Advantageously, in accordance with some embodiments, the base 12 can provide sufficient structural strength, as well as a large surface area that can be exposed to the subdermal cavity, thereby maximizing the initial absorption rate of fluid by the base 12. The surface texture, absorption surface area, fluid collection component thickness, and other such variables can be modified to provide a desired absorption profile. For example, as shown in FIG. 3, the fluid collection component 28 can have a generally constant cross-sectional thickness

In some embodiments, the apertures 30 can be used to allow the clinician to suture the tissue expander in place during implantation such that the tissue expander does not slide or rotate out of place after implantation. In such embodiments, the base 12 can provide appropriate structural durability. For example, the base 12 can incorporate the tear-resistant material component 24 to provide the base 12 with sufficient structural strength to function as a suture tab. Advantageously, when the fluid collection component is included in the base 12 adjacent to the apertures 30, the extracellular fluid resulting from suturing process can be absorbed and wicked away by the fluid collection component 28.

Additionally, as shown in FIGS. 1 and 2, the base 12 can extend and/or expand out beyond a perimeter or periphery of the expander shell 14, enabling the fluid collection component 28 to wick away the extracellular fluid from areas of the expander shell 14, such as areas of the subdermal cavity that are further away from the tissue expander.

The fluid collection component 28 can be configured as a layer of absorptive material, one or more channels extending along the tissue expander 10 (for example, along the base 12), as pockets or compartments of absorptive material, or combinations of the same. For example, the fluid collection component can comprise a ring that extends around a perimeter of the tissue expander 10 between the base 12 and the expandable shell 14, such as extending partially or entirely around the expandable shell 14 in a circumferential gap 34, as illustrated in FIG. 2. Various other configurations can be implemented in addition to those discussed and shown herein.

In some embodiments, the fluid collection component 28 may be included on or in the expandable shell 14. The fluid collection component 28 may be on an exterior surface of the expandable shell 14 that is exposed to the body upon implantation or an interior surface of the expandable shell 14 that is not exposed to the body upon implantation. In some embodiments, the fluid collection component 28 may be provided at an interface between the base 12 and the expandable shell 14, in particular where the base 12 attaches to the anterior portion 16 and/or the posterior portion 18 (e.g., in the gap 34).

The fluid collection component 28 comprise a foam-like absorbing matrix material, in some embodiments. In some embodiments, the fluid collection component 28 can include a fibrous mesh, an open cell matrix foam, an expandable elastomeric matrix or other similar materials. In some embodiments, the fluid collection component 28 may include natural or synthetic fibrous structures such as, for example, those formed of polyester, cellulose, cotton, wool, or hemp, that are configured alone or in combination with each other into yarns or sheets or other absorbable structures.

In some embodiments, the fluid collection component 28 may include a solute embedded within a matrix defining boundaries of a plurality of chambers within the matrix. The solute, in such embodiments, may provide for an osmotic gradient such that the extracellular fluid can be absorbed into the matrix. The solute may be, for example, a salt such as sodium chloride or a sugar. Other examples of the solute include, but are not limited to, potassium or calcium chloride, iodide, bromide, carbonate, bicarbonate, sulfate, sulfite, phosphate, or acetate of sodium, potassium or calcium, and combinations thereof, or water soluble organic species sch as amino acids, conjugate bases of amino acids, carboxylic acids, conjugate bases of carboxylic acids, (e.g. pyruvate, succinate, etc.), alcohols comprising 2 or more carbon atoms, monomeric and dimeric sugars, and peptides.

In some embodiments, the fluid collection component 28 may further include hydrogel particles configured to retain fluid absorbed by the fluid collection component 28.

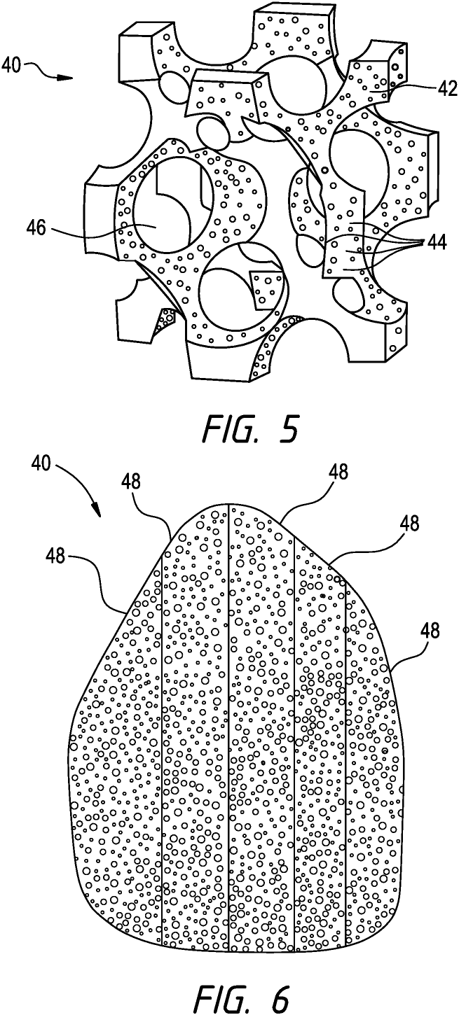

Referring to FIGS. 4 and 5, in some embodiments, the fluid collection component 28 may comprise a matrix 40 having walls 42 that define boundaries of multiple independent chambers 46 that are distributed throughout the matrix 40. The multiple chambers 46 can be interconnected in an open cell configuration. Each chamber 46 can be in fluid communication with other chambers 46 as the walls 42 partially separate adjacent chambers 46. The walls 42 can include or be constructed of an elastomer or other flexible material that is a semi-permeable membrane that selectively allows diffusion of certain substances. According to some embodiments, the matrix 40 can be a foam matrix.

According to some embodiments, the matrix 40 can include granules of a solute 44 embedded within cells in the elastomer of the walls 42. At least some of the granules of solute 44 can be fully encapsulated within the walls 42.

In some embodiments, the walls 42 can be semipermeable, and the presence of the granules of solute 44 can establish an osmotic gradient when in an aqueous environment and with respect to fluid within the chambers 46 and/or fluid outside of the device 10. The cells with the solute 44 in the walls 42 can selectively allow diffusion of certain substances, such as saline or body fluids, into the chambers 46 and toward the granules of solute 44.

For example, as the fluid diffuses through the walls 42, the cells containing the granules of solute 44 can absorb the fluid and cause the cells to expand which fill the chambers 46 and cause the matrix 40 to expand from the collapsed state to an expanded state. According to some embodiments, expansion can continue until the device 10 reaches osmotic equilibrium with the external environment in the subdermal cavity in which the tissue expander is implanted or until another constraint is applied. In some embodiments, the once expanded to a certain size, the cells may seal themselves, thereby preventing loss of fluid from the cell. Advantageously, such self-sealing cells can prevent the fluid collected by the fluid collection component from getting squeezed out when pressure is applied on the implant, e.g., during expansion of the tissue expander, or during the procedure for removing the tissue expander.

In accordance with some embodiments disclosed herein is the realization that the amount of extracellular fluid that may be produced following the implantation of the tissue expander can depend on the patient and the tissue area being treated. Further, in some embodiments, the material properties of the fluid collection component 28 may be adjusted to allow more or less fluid to be absorbed in the matrix 40.

For example, some embodiments can be configured to provide a higher concentration of the solute 44 in order to help establish a steeper slope for the osmotic gradient, thereby inducing more rapid diffusion of the extracellular fluid into the fluid collection component 28. A solution comprising solute therein can be characterized as having an “osmotic concentration” or “osmolarity,” which is defined as the total number of solute particles per liter of solution. In some embodiments, the permeability of the walls 42 can provide higher and/or lower permeability sections in order to alter the rate of diffusion of the extracellular fluid into the matrix 40, thereby governing the rate of fluid absorption.

When the tissue expander is implanted, the trauma caused to the surrounding tissue during the implantation results in formation of extracellular fluid in the cavity in which the tissue expander is implanted. Because the rate of formation of the extracellular fluid may be greater than the capacity of the lymphatic system to remove the extracellular fluid, the fluid may accumulate in the cavity at a certain rate following the implantation procedure. Over time, as the tissue heals, the rate of formation of the extracellular fluid is reduced as the tissue heals and the lymphatic system picks up the extracellular fluid. Advantageously, in some embodiments, the matrix can be structured such that a rate of diffusion of the extracellular fluid into the matrix 40 is initially relatively high, and the rate of diffusion decreases over time. Thus, the tissue expander 10 can provide a high absorptivity of extracellular fluid upon implantation and slowly decrease over time as the lymphatic system balances its drainage against the body’s production of extracellular fluid.

For example, in some embodiments, as the solute gets dissolved in the extracellular fluid absorbed by the matrix 40, the osmotic gradient reduces, thereby reducing the rate of diffusion of the extracellular fluid into the matrix, eventually reaching an equilibrium.

The amount and rate of formation of extracellular fluid produced after a surgical procedure typically can depend on the size of the tissue. For example, the extracellular fluid in the inframammary fold in case of a breast reconstruction procedure can be generated at a faster rate than in case of an intraoral, a periocular, or a cheek reconstruction procedure. Thus, depending on the size of the tissue, the initial amount of solute 44 in the matrix 40 can be adjusted to provide an appropriate equilibrium point.

In accordance with some embodiments, the initial concentration and amount of solute in fluid collection component 28 prior to implantation can be preselected in order to achieve a final diluted concentration that can be equal to or in slight excess of a solute concentration in the extracellular fluid.

For example, the final solute concentration (osmolarity) in the expanded state can be about 0% to about 10%, about 5% to about 15%, about 10% to about 20%, or about 15% to about 25% greater than the total solute concentration (osmolarity) in the extracellular fluid.

According to some embodiments, the matrix 40 can include a fluid permeation profile that characterizes the manner and conditions for allowing diffusion of materials therethrough. According to some embodiments, the matrix 40 includes a fluid permeation profile whereby fluid permeation occurs while within the body of the patient.

For example, the fluid permeation can occur at temperatures between about 35° C. and about 41° C., such as between about 36.5° C. and about 37.5° C. The fluid permeation profile of the matrix 40 can be controlled, for example, by configuration of the material composition of the matrix 40.

Further, in accordance with some embodiments, the material composition of the matrix 40 can be configured with respect to characteristics including, hydrophilicity, hydrophobicity, porosity and pore size, elastic modulus, thickness, surface area, and concentration and amount of solute 44 encapsulated within matrix 40. Accordingly, the fluid collection component 28 can be designed to absorb extracellular fluid until the rate of formation of the extracellular fluid is in equilibrium with the rate of uptake by the lymphatic system, as described elsewhere herein.

According to some embodiments, the solute 44 can be provided as granules. The size and quantity of granules can be selected to achieve a particular rate of absorption of extracellular fluid.

For example, in some embodiments, the size of the granules can define a total volume of extracellular fluid absorbed within walls 42. As water permeates into these encapsulated volumes the granules can dissolve and the volume of absorbed fluid will increase.

The size of the granule can be correlated to the concentration of the solute 44 within the absorbed volume, which will affect the osmotic gradient between the absorbed volume and the extracellular fluid in the subdermal cavity. Thus, smaller granules will generally result in less swelling while larger granules will generally result in more swelling.

Similarly, in accordance with some embodiments, a larger concentration of solute 44 (e.g., above 10%, above 15%, above 20%, or above 25%) can be encapsulated within walls 42 and generally result in more absorption while lower concentrations (e.g., let them 10%, less than 5%, or less than 3%) can generally result in less absorption.

Further, the matrix 40 of the tissue expander can be configured such that the solute 44 includes granules within a size range of about 100 to about 500 mesh, about 200 to about 400 mesh, about 250 to about 350 mesh, about 275 to about 325 mesh, or about 300 mesh.

Moreover, the solute 44 of the matrix 40 can optionally include biocompatible materials capable of establishing an osmotic gradient, including a salt, such as sodium chloride.

In some embodiments, solute 44 can be an ionic solute selected from the group consisting of sodium, potassium, calcium, chloride, iodide, bromide, carbonate, bicarbonate, sulfate, sulfite, phosphate, acetate, and combinations thereof. In some embodiments, solute 44 can be a water-soluble organic species. Water-soluble organic species include, but are not limited to amino acids, conjugate bases of amino acids, carboxylic acids, conjugate bases of carboxylic acids, (e.g. pyruvate, succinate, etc.), alcohols comprising 2 or more carbon atoms, monomeric and dimeric sugars, and peptides.

The base 12, or portions thereof, can be formed of silicone rubber, a laminate of various forms of silicone, silicone copolymers, polyurethane, and various other elastomers in various combinations. In some embodiments, the elastomer component comprises a biocompatible, silicone elastomer material.

For example, the elastomer component may comprise any suitable silicone elastomeric material. Suitable silicone elastomers include, but are not limited to, homopolymers such as polydimethylsiloxane or polymethylvinylsiloxane, or copolymers such as copolymers of diphenylsiloxane and dimethylsiloxane. The silicone elastomer can be cured by conventional means, for example, by using a polysiloxane crosslinker containing silicone-bonded hydrogen atoms with a vinyl containing siloxane elastomer and a platinum catalyst.

According to some embodiments, the granules of solute 44 can be mixed with an elastomer that is included in the walls 42 of the matrix 40. The elastomer can include one or more biocompatible materials capable of being semi-permeable.

In some embodiments, the elastomer can include silicone. The solute can be mixed with the elastomer at a ratio of about 1% to about 20% by weight, for example about 5% to about 10% by weight. The concentration of solute in the elastomer can be selected to achieve a particular rate of expansion and/or target characteristics of the device 10 in the expanded state. For example, a human body can have solutes in extracellular fluids that include sodium, potassium, calcium, chloride, phosphate, and bicarbonate. In addition, small peptides or glucose can be present. These solutes can be present in a total concentration in a range from about 240 mM to about 272 mM. The concentration of the solute 44 in the elastomer can be configured in order to achieve a final concentration of solute 44 after the extracellular fluid has been absorbed into the base 12 that is similar to that of extracellular fluids in a human body.

According to some embodiments, the mixture can be formed into the matrix 40 using a manufacturing or assembly process that creates the chambers 46. For example, the mixture can be subjected to an open cell foaming process, such as air entrapment, thermal decomposition with a blowing agent, or porogen bead fusion technology. By further example, the matrix 40 could be molded, 3-D printed, and/or be a two-dimensional mesh that can be folded or stacked into 3-D shapes.

The elastomer of the mixture can be cured and formed approximately into the shape of the matrix 40 that is desired when the fluid is absorbed within the fluid collection component 28. The permeability of the matrix 40 can be programmed or altered based on certain parameters. For example, selections can be made with regard to the composition of the polymer, molecular weight, functional groups, wall thickness, chamber or pore size, and/or surface area of the matrix.

According to some embodiments, the matrix 40 can be a single monolith that can be integrally formed by a network of walls 42. The monolithic structure can be contained within the base 12.

Referring to FIG. 6, according to some embodiments, the matrix 40 can include a plurality of separate, distinct segments 48 that are each smaller than the whole matrix 40. The segments 48 can facilitate manipulation of the matrix 40, including implantation and removal of the tissue expander 10, through arrangement of the individual segments 48. For example, the matrix 40 can have a composite shape that is defined by the arrangement of individual segments 48. The composite shape can be achieved while the segments 48 are arranged within the base 12. The segments 48 can be formed by a series of cuts or partitions into the matrix 40 after formation of the matrix 40 as a monolithic structure. The cuts can be along intersecting or non-intersecting planes. For example, the segments can extend longitudinally and be divided along parallel planes. The matrix 40 can be shaped during and/or after curing. According to some embodiments, the matrix 40 can be divided or formed into segments 48.

Additionally, two or more of the segments can have different osmotic gradient properties, such as solute concentration, which would allow for different amounts and rates of extracellular fluid to be absorbed into the segments within the base 12.

For example, portions of the base 12 further away from the inframammary fold may have a relatively higher solute concentration to allow the absorbed extracellular fluid to be retained in portions away from the areas where the rate of formation of the extracellular fluid is higher so as to provide a wicking effect. According to some embodiments, a concentration gradient of the solute can be provided such that the absorbed extracellular fluid can be retained and isolated in a central region of the base 12.

In some embodiments, the fluid collected and retained by the fluid collection component can provide at least a portion of the intended expansion volume of the tissue expander. In such embodiments, the increase in the volume of the matrix of the fluid collection component causes corresponding increase in the volume of the expandable shell. Such configuration is achieved by, for example, providing the fluid collection component on an interior or exterior surface of the expandable shell. Alternatively, the fluid collection component can be configured to transport (i.e., wick away) the collected extracellular fluid and retain the extracellular fluid in a region interior to the expandable shell.

In some embodiments, the fluid collection component can be separated from the expandable shell such that the fluid collected and retained by the fluid collection component is not exposed or mixed with the fluid used for expanding the expandable shell. For example, a water impermeable barrier may be disposed between the fluid collection component and the internal volume of the expandable shell.

In accordance with some embodiments, as shown in FIG. 7, the base 12 of the tissue expander 10 can comprise a plurality of tabs 92, arrange about the periphery or rim 20. The individual tabs 92 can be movable relative to the expandable shell 14. For example, the tabs 92 can be selectively folded against or extended away from the expandable shell 14 in order to extend through slots of a wrap 98, such as an Acelluar Dermal Matrix (ADM). The wrap 98 is commonly used in breast reconstruction, can be used with the tissue expander 10. Typically, the wrap 98 can be provided separately in sheet form and an inferior portion of the expander 10 can be wrapped with the wrap 98 by the clinician on a back table, e.g., before implantation, and the assembly is then implanted. In such embodiments, for example, a portion of the base of the tissue expander, such as for example, a suture tab, may be extended out from the wrap 98 to enable the fluid collection component to absorb fluid that is positioned adjacent to the wrap 98 (using tabs 92 that are stowed or folded against the expandable shell 14 within the wrap 98) or wick away the fluid adjacent the wrap 98 (using tabs 92 that are extended away from the expandable shell 14 and passing through slots of the wrap 98). Thus, the tabs 92 of the tissue expander 10 can be used for both fluid absorption purposes and to couple the tissue expander 10 using sutures 104 in order to secure the tabs 92 relative to the tissue.

In practice, the tissue expander described herein may be used for expanding soft tissue in a body of a living subject, e.g., for breast reconstruction following a mastectomy. In accordance with some embodiments of the present disclosure, a method for expanding soft tissue in a body of a living subject may include forming a subdermal cavity at a site where the soft tissue is to be expanded, and implanting the tissue expander described herein in the subdermal cavity.

For example, the tissue expander may comprise an expandable shell having an anterior portion, a posterior portion and a perimeter. The posterior portion comprises a fluid collection component configured to absorb extracellular fluid adjacent the tissue expander following the implantation of the tissue expander.

In some embodiments, when implanting, the tissue expander can be positioned within the subdermal cavity such that the posterior portion contacts an inferior portion of the subdermal cavity and the extracellular fluid from the inferior portion of the subdermal cavity can be absorbed into the fluid collection component.

In some embodiments, the posterior portion of the tissue expander includes a tear-resistant material and suture tabs formed in the tear-resistant material. A portion of the perimeter of the posterior portion may thus be sutured to the tissue site where the tissue expander is inserted so as to hold the tissue expander in place and prevent it from sliding or rotating within the subdermal cavity.

Following the implantation of the tissue expander, the expandable shell can be gradually expanded, e.g., by filling the expandable shell with a fluid, to expand the soft tissue. In some embodiments, the expandable shell can be periodically filled with small amounts of fluid to reduce or minimize the pain and discomfort to the subject that may be caused by sudden expansion the soft tissue.

In some embodiments, when the desired expansion of the soft tissue is reached, the tissue expander may be surgically removed, and a permanent implant, e.g., in case of breast reconstruction, may be inserted into the subdermal cavity to complete the procedure.

Some embodiments described herein also provide a method of reducing the amount of extracellular fluid adjacent a tissue expander upon implantation of the tissue expander in a body of a living subject. The method may include positioning a tissue expander described herein within a subdermal cavity of the subject such that a posterior portion of the tissue expander contacts an inferior portion of the subdermal cavity and the extracellular fluid from the inferior portion of the subdermal cavity can be absorbed into the fluid collection component of the tissue expander.

Thus, the devices and methods disclosed herein can not only facilitate reduction in accumulation of extracellular fluid in a subdermal cavity following a surgical procedure, but can beneficially utilize such fluid for expanding the tissue expander by collecting and retaining the extracellular fluid in a fluid absorbing or collecting component.

The fluid collecting component can be coupled, attached, integral with (such as a single, continuous form, material, or part), or connected to any portion or structure of the tissue expander.

The devices disclosed herein may be used for reducing the accumulation of extracellular fluid following a procedure used for expanding soft tissue in a living subject. Further, the size of the tissue expander can be monitored by a clinician and adjusted by either adding or removing fluid from the tissue expander to achieve a desired expansion rate for the patient.

Accordingly then, some embodiments disclosed herein can provide substantial benefits over prior devices that do not allow a patient to benefit from a dynamic tissue expander that helps to preserve patient health, avoid complications, reduce potential surgical interventions, and potentially allow the clinician to perform fewer invasive procedures as the tissue expander and/or one or more of its components absorbs or collects extracellular tissue to thereby expand in size.

The tissue expander disclosed herein may include an expandable shell and a fluid collection component connected, affixed or otherwise attached to the expandable shell. The fluid collection component can be configured to absorb or collect extracellular fluid adjacent the tissue expander after the tissue expander has been implanted into a subdermal cavity, they are the soft tissue case to be expanded. The tissue expander disclosed herein reduces the incidence of seroma or infections by collecting, retaining, and isolating the extracellular fluid away from the subdermal cavity.

The method for expanding a soft tissue in a body of a living subject disclosed herein may include implanting a tissue expander disclosed herein into a subdermal cavity such that a posterior portion of the tissue expander contacts an inferior portion of the subdermal cavity so as to collect the extracellular fluid in the subdermal cavity, and retain and isolate the collected extracellular fluid into the tissue expander.

A method for reducing the amount of extracellular fluid accumulating in a subdermal cavity following the implantation of a tissue expander may include positioning a tissue expander disclosed herein such that a posterior portion of the tissue expander contacts a portion of the subdermal cavity where the extracellular fluid accumulates such that the accumulated extracellular fluid can be collected by the tissue expander, and retained and isolated the collected extracellular fluid into the tissue expander.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A tissue expander comprising: an expandable shell; and a fluid collection component coupled to the expandable shell and configured to absorb extracellular fluid adjacent the tissue expander upon implantation.

Clause 2. The tissue expander of Clause 1, further comprising a base coupled to a posterior of the expandable shell.

Clause 3. The tissue expander of Clause 2, wherein the at least a portion of the base extends radially beyond a periphery of the expandable shell.

Clause 4. The tissue expander of Clause 2, wherein the fluid collection component is carried by the base.

Clause 5. The tissue expander of Clause 2, wherein the base comprises a circumferential flange.

Clause 6. The tissue expander of Clause 2, wherein the base comprises a plurality of suture tabs.

Clause 7. The tissue expander of Clause 2, wherein the base comprises a layered structure having a tear-resistant portion and the fluid collection component.

Clause 8. The tissue expander of any of the preceding Clauses, wherein the fluid collection component comprises a fibrous mesh.

Clause 9. The tissue expander of any of the preceding Clauses, wherein the fluid collection component is carried within the expandable shell.

Clause 10. The tissue expander of any of the preceding Clauses, wherein the fluid collection component is carried on an outer region of the expandable shell.

Clause 11. The tissue expander of any of the preceding Clauses, wherein the fluid collection component is incorporated into a layer of the expandable shell.

Clause 12. The tissue expander of any of the preceding Clauses, wherein the fluid collection component is incorporated into only a circumferential region of the expandable shell.

Clause 13. The tissue expander of any of the preceding Clauses, wherein the fluid collection component is configured to absorb and retain the extracellular fluid.

Clause 14. The tissue expander of any of the preceding Clauses, wherein the fluid collection component comprises an open cell matrix foam.

Clause 15. The tissue expander of any of the preceding Clauses, wherein the fluid collection component comprises hydrogel particles configured to retain fluid.

Clause 16. The tissue expander of any of the preceding Clauses, wherein the fluid collection component comprises salt particles.

Clause 17. The tissue expander of any of the preceding Clauses, wherein the fluid collection component comprises an expandable elastomeric matrix and granules of a solute embedded within the matrix, wherein the matrix defines boundaries of a plurality of chambers within the matrix.

Clause 18. The tissue expander of Clause 17, wherein the expandable elastomeric matrix comprises a foamed elastomer.

Clause 19. The tissue expander of any one of Clauses 17 to 18, wherein the expandable elastomeric matrix comprises silicone.

Clause 20. The tissue expander of any one of Clauses 17-19, wherein the granules of solute comprise sodium chloride.

Clause 21. The tissue expander of any one of Clauses 17-20, wherein the chambers are fluidly interconnected as open cells of the matrix.

Clause 22. The tissue expander of any of the preceding Clauses, wherein the expandable shell has an anterior portion, a posterior portion and a perimeter, wherein the anterior portion comprises the fluid collection component.

Clause 23. The tissue expander of Clause 22, wherein the at least a portion of the posterior portion of the tissue expander comprises a laminate including the fluid collection component and a tear-resistant material.

Clause 24. The tissue expander of Clause 23, wherein the laminate extends from the perimeter of the tissue expander, thereby forming a ring around the posterior portion of the tissue expander.

Clause 25. The tissue expander of Clause 24, wherein the ring is configured to wick away the extracellular fluid from a tissue adjacent the tissue expander.

Clause 26. The tissue expander of any of the preceding Clauses, further comprising an acelluar dermal matrix.

Clause 27. A method for expanding a soft tissue in a body of a living subject, the method comprising: forming a subdermal cavity at a site where the soft tissue is to be expanded; and implanting a tissue expander in the subdermal cavity, the tissue expander comprising an expandable shell having an anterior portion, a posterior portion and a perimeter, wherein the posterior portion comprises a fluid collection component configured to absorb extracellular fluid adjacent the tissue expander upon implantation.

Clause 28. The method of Clause 27, wherein implanting the tissue expander comprises positioning the tissue expander within the subdermal cavity such that the posterior portion contacts an inferior portion of the subdermal cavity and extracellular fluid from the inferior portion of the subdermal cavity is absorbed into the fluid collection component.

Clause 29. The method of any one of Clauses 27 to 28, wherein implanting the tissue expander comprises inserting the tissue expander in the subdermal cavity and suturing a portion of a perimeter thereof to an inferior portion of the subdermal cavity.

Clause 30. The method of any one of Clauses 27-29, further comprising permitting expansion of the expandable shell upon implantation of the tissue expander so as to expand the soft tissue.

Clause 31. The method of Clause 30, wherein the permitting expansion comprises expanding the expandable shell by filling the expandable shell with a fluid.

Clause 32. A method of reducing an amount of extracellular fluid adjacent a tissue expander upon implantation of the tissue expander, the method comprising positioning the tissue expander having a posterior portion comprising a fluid collection component configured to absorb extracellular fluid within a subdermal cavity such that the posterior portion contacts an inferior portion of the subdermal cavity to permit the extracellular fluid from the inferior portion of the subdermal cavity to be absorbed into the fluid collection component.

### Further Considerations

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. A tissue expander comprising:
an expandable shell; and
a fluid collection component coupled to the expandable shell and configured to absorb extracellular fluid adjacent the tissue expander upon implantation.

2. The tissue expander of claim 1, further comprising a base coupled to a posterior of the expandable shell.

3. The tissue expander of claim 2, wherein the at least a portion of the base extends radially beyond a periphery of the expandable shell.

4. The tissue expander of claim 2, wherein the fluid collection component is carried by the base.

5. The tissue expander of claim 2, wherein the base comprises a circumferential flange.

6. The tissue expander of claim 2, wherein the base comprises a plurality of suture tabs.

7. The tissue expander of claim 2, wherein the base comprises a layered structure having a tear-resistant portion and the fluid collection component.

8. The tissue expander of claim 1, wherein the fluid collection component comprises a fibrous mesh.

9. The tissue expander of claim 1, wherein the fluid collection component comprises an open cell matrix foam.

10. The tissue expander of claim 1, wherein the fluid collection component comprises hydrogel particles configured to retain fluid.

11. The tissue expander of claim 1, wherein the fluid collection component comprises salt particles.

12. The tissue expander of claim 1, wherein the fluid collection component comprises an expandable elastomeric matrix and granules of a solute embedded within the matrix, wherein the matrix defines boundaries of a plurality of chambers within the matrix.

13. The tissue expander of claim 1, wherein the expandable shell has an anterior portion, a posterior portion and a perimeter, wherein the anterior portion comprises the fluid collection component.

14. The tissue expander of claim 13, wherein the at least a portion of the posterior portion of the tissue expander comprises a laminate including the fluid collection component and a tear-resistant material.

15. The tissue expander of claim 14, wherein the laminate extends from the perimeter of the tissue expander, thereby forming a ring around the posterior portion of the tissue expander.

16. The tissue expander of claim 15, wherein the ring is configured to wick away the extracellular fluid from a tissue adjacent the tissue expander.

17. A method for expanding a soft tissue in a body of a living subject, the method comprising:
forming a subdermal cavity at a site where the soft tissue is to be expanded; and
implanting a tissue expander in the subdermal cavity, the tissue expander comprising an expandable shell having an anterior portion, a posterior portion and a perimeter,
wherein the posterior portion comprises a fluid collection component configured to absorb extracellular fluid adjacent the tissue expander upon implantation.

18. The method of claim 17, wherein implanting the tissue expander comprises positioning the tissue expander within the subdermal cavity such that the posterior portion contacts an inferior portion of the subdermal cavity and extracellular fluid from the inferior portion of the subdermal cavity is absorbed into the fluid collection component.

19. The method of claim 17, wherein implanting the tissue expander comprises inserting the tissue expander in the subdermal cavity and suturing a portion of a perimeter thereof to an inferior portion of the subdermal cavity.

20. The method of claim 17, further comprising permitting expansion of the expandable shell upon implantation of the tissue expander so as to expand the soft tissue.

21. The method of claim 20, wherein the permitting expansion comprises expanding the expandable shell by filling the expandable shell with a fluid.

22. A method of reducing an amount of extracellular fluid adjacent a tissue expander upon implantation of the tissue expander, the method comprising positioning the tissue expander having a posterior portion comprising a fluid collection component configured to absorb extracellular fluid within a subdermal cavity such that the posterior portion contacts an inferior portion of the subdermal cavity to permit the extracellular fluid from the inferior portion of the subdermal cavity to be absorbed into the fluid collection component.
